(19) United States
(12) Patent Application Publication
(10) Pub. No.: US 2016/0106903 A1
(43) Pub. Date: Apr. 21, 2016

(54) Fluid Pump Systems, and Related Methods for Pumping Biological Fluids

(71) Applicant: HESTIAMEDTECH AB, Göteborg (SE)

(72) Inventors: Per Nilsson, Hönö (SE); Rolf Andersson, Bro (SE)

(21) Appl. No.: 14/787,425

(22) PCT Filed: Apr. 29, 2014
(86) PCT No.: PCT/EP2014/058750

§ 371 Date: Oct. 27, 2015

Related U.S. Application Data
(60) Provisional application No. 61/817,066, filed on Apr. 29, 2013.

(51) Int. Cl. A61M 1/26; F04B 23/02; F04B 43/02; A61M 1/34; F04B 43/06

(52) U.S. Cl. CPC A61M 1/267; A61M 1/342; F04B 43/06; F04B 43/026; F04B 23/02; A61M 2202/30; A61M 2202/07

(57) ABSTRACT

The present invention relates to a fluid pump system (64) having a fluid reservoir (21) comprising a first cavity (29) and a second cavity (30) in fluidic communication with each other and adapted to accommodate a biological fluid (7). The fluid reservoir (21) is connected to a fluid-container system (27) and a target (5) is in fluidic communication with the second cavity (30). A pressurization system is adapted to generate a first pressure below ambient pressure in the first cavity (29) and generate a second pressure in the first cavity (29). The second pressure is also applied to the second cavity (30). The fluid pump system (64) thereby causes flow of the biological fluid (7) from the fluid-container system (27) into the first cavity (29), from the first cavity (29) into the second cavity (30), and from the second cavity (30) to the target (5).

### TECHNICAL FIELD

The invention generally relates to fluid pump systems, and related methods of use thereof, particularly methods and fluid pump systems for pumping biological fluids. The present invention may be particularly suitable for use with blood pumps, e.g. extracorporeal blood pumps, such as those used in dialysis machines and heart-lung machines, and for use during the production of various compounds and substances, e.g. proteins, such as insulin, and for use in connection with bioreactors. The present invention may additionally have other applications where its characteristics are desirable.

### BACKGROUND

Because of the unique biological environment in which they operate, blood pumps must satisfy very exacting operational requirements that relate primarily to the prevention of blood damage and the prevention of air being transferred to the recipient (transferred air can result in emboli, so called air embolism) and not risk contaminating the blood with foreign matter, such as silicone. Pumps made for the production of various compounds and substances, e.g. proteins, hormones, antibodies, biological drugs, complex drugs, vaccines, biopharmaceuticals, etc., can be sensitive to e.g. shear and high pressure during production.

Correspondingly, pumps made for bioreactors have high demands on e.g. controlled pressure, sometimes very low pressures, prevention of air bubbles and contamination of the media with foreign matter. Pumps in bioreactors can have different functions e.g. pumping culture fluid, other fluids and cells in and out of e.g. a container in the bioreactor.

Blood pumps impart fluid motion to the blood that is not ordinarily experienced during normal biological processes. This fluid motion may jeopardize the integrity of the blood cells and present an excessive risk that hemolysis—the damage of blood cell membranes—will occur because of the excessive fluid shear and frictional forces experienced by the pumped blood. Damage to the blood results from mechanical action, turbulence or shear exerted on various blood elements, especially the red blood cells, but also denaturation of proteins is a problem in the field. Hemolysis of blood normally occurs in turbulent areas within a pump, primarily on foreign surfaces. Blood cell integrity in the pumped blood is also at risk because of the heat generated by friction between the moving parts of traditional blood pumps. Frictional energy may result ultimately in thrombosis—the undesirable clotting or coagulation of blood. It may also increase the potential for the formation of protein deposits within the pump structure or within the blood. Thrombosis or clotting can also be caused by stagnation of blood in the pump. If the flow undergoes a low or zero velocity region, it may experience thrombosis or clotting, where blood resides on the pump structure. Such low or zero velocity regions can, for example, be found in secondary blood paths in the pump. As the thrombus builds up, a section or large clot may break off and embolize (thromboembolism) in the blood stream. If the clot occludes a blood vessel that enters the brain or other sensitive areas, very serious conditions may develop, such as profound organ dysfunction and stroke.

Typical pumps often have the disadvantage that they may cause cavitation, thereby creating bubbles. These bubbles may result in air emboli. Cavitation occurs during pumping of a fluid, if, in the suction phase, a pressure below ambient pressure is produced which is strong enough to make the static pressure of the fluid drop to the vapour pressure of the fluid. The fluid then locally passes into gaseous form. Blood pumps normally pump small volumes at a high frequency to achieve a sufficient flow; they can be said to have a small displacement, i.e. pump a small volume per cycle. This means the pump is using a pressure in the suction phase, which is significantly lower than the ambient pressure and therefore have a tendency to produce cavitation. Blood pumps also generally generate high shear forces which may destroy sensitive components of the blood.

Extracorporeal blood pumps or in vivo heart assist devices comprise various designs, including peristaltic roller pumps, centrifugal pumps, axial floe pumps, pneumatic chamber pumps, and hydraulic chamber pumps. These pumps vary widely in cost and efficacy, and their uses vary from supporting a patient on a heart/lung machine to assisting the human heart as a bridge to transplant or replace the human heart.

Both roller and centrifugal pumps tend to cause hemolysis in that they break up red blood cells. Roller pumps cause damage to blood elements as a result of the intermittent mechanical shearing caused by the occlusion of the tubing by the pump. This may lead to haemolysis and release of vasoactive substances. Roller compression of the tubing causing the tubing to be pinched between the roller and raceway can produce a breakdown in the tubing wall and release of small plastic particles into the fluid stream, and risk of e.g. particulate embolism or other detrimental conditions. Centrifugal pumps rupture the cells during high-speed circulation. Hemolysis decreases further the already reduced hematocrit, if present, of the extracorporeal perfusate. Hemolysis, if material, has to be corrected by infusion of foreign packed blood cells or blood transfusions to maintain physiological respiration at safe levels. Rotary pumps for pumping blood can cause severe damage in the blood, i.e. hemolysis. The higher or lesser extent at which the blood is damaged will depend on many factors, one of the main factors being the high shear forces or stresses affecting the red cells and platelets, such stresses appearing in zones between pump components with relative movements and close to each other or, worst, in contact with each other.

According to Publication No. 85-2185; 1985; from the National Institute of Health (NIH), entitled “Guidelines for Blood-Material Interactions”, it is generally accepted that the quantity of red cells and platelets damaged by shear stresses depends on the intensity or magnitude of the stresses and the period of time the red cell and/or platelet is exposed to the stresses for a determined quantity of hematocrit. The hematocrit is the volume percentage of red cells in the blood.

Blood pumps can be categorized as producing pulsatile or non-pulsatile flows. Non-pulsatile pumps generally do not require valve systems, but do require rotating shafts passing through bearings and seals. Pulsatile pumps universally require valves (mechanical or tissue). A number of designs have been developed to introduce a pulsatile component to extracorporeal circulation. These designs generally fall into two categories. The first category includes those devices that combine a roller or centrifugal pump with an additional device that periodically compresses the tube through which the blood or cardioplegia flows. A second category includes devices in which the pump itself is used to produce a pulsatile flow. Both of these types of designs, however, are limited in their ability to produce a pulsatile flow of desired characteristics while still maintaining a desired average flow rate. Motor driven syringe pumps have disadvantages in that the syringe has a small volume and the process has to be stopped to refill the syringe.

Several of the problems described above for blood pumps may also be present in other applications using pumps in the life sciences field. For example, today's production of sensitive biological fluids (e.g. fluid comprising insulin and/or other biologicals) face a problem with breakdown of the product when/if pumping the fluids with the currently available pumps and there is a need for a pumping system that has a very low degree of shear forces and good pressure control.

Bioreactors can be used for various types of production of biological matter, e.g. production of tissue-engineered biocompatible tissues or organs.

One way of doing this is to first remove all the cells from an organ or tissue by decellularization where one is stripping away the donor cells and a protein scaffold is left behind. The next step can be to repopulate the scaffold again—recellularization, by using e.g. autologous stem cells from the recipient i.e. immunologically matched cells.

Air bubbles and particles that adhere to the tissues or organs can perturb these processes.

The present invention may address previous shortcomings in the art by providing fluid reservoirs, related systems comprising the same, and related methods of use thereof.

### SUMMARY

A first aspect of the present invention comprises a method of pumping a biological fluid to a target. The method comprises providing a fluid pump system comprising: a fluid reservoir comprising at least one first cavity and at least one second cavity in fluid communication with each other and configured to accommodate the biological fluid. The at least one second cavity is in fluid communication with the target. The fluid pump system also comprises a fluid-container system in fluid communication with the at least one first cavity in the fluid reservoir, and a pressurization system configured to generate a pressure in the at least one first cavity and the at least one second cavity. The method also comprises generating a first pressure below ambient pressure in the at least one first cavity using the pressurization system, thereby causing the biological fluid to flow from the fluid-container system into the at least one first cavity and generating a second pressure in the at least one first cavity using the pressurization system, thereby causing the biological fluid in the at least one first cavity to flow into the at least one second cavity. The method further comprises applying the second pressure to the at least one second cavity, thereby pumping the biological fluid to the target.

A second aspect of the present invention relates to a fluid pump system comprising a fluid reservoir comprising at least one first cavity and at least one second cavity in fluid communication with each other and configured to accommodate a biological fluid. The at least one second cavity is in fluid communication with the target. The fluid pump system also comprises a fluid-container system in fluid communication with the at least one first cavity in the fluid reservoir. The fluid pump system further comprises a pressurization system configured to generate a first pressure below ambient pressure in the at least one first cavity, thereby causing the biological fluid to flow from the fluid-container system to the at least one first cavity. The pressurization system is also configured to generate a second pressure in the at least one first cavity, thereby causing the biological fluid in the at least one first cavity to flow into the at least one second cavity. The pressurization system is further configured to apply the second pressure to the at least one second cavity, thereby pumping the biological fluid out from the at least one second cavity.

A further aspect of the present invention comprises a method of pumping a biological fluid to a biological target and/or machine, the method comprising: providing a pump system comprising: a fluid reservoir comprising a first cavity at least partially defined by a first deformable membrane to provide a first volume or second chamber; a fluid-container system in fluid communication with said fluid reservoir; and a pressurization system configured to generate a pressure in said first cavity; generating a first pressure configured to deform said first membrane to expand said first volume or second chamber of said first cavity using said pressurization system, thereby causing said biological fluid to flow from said fluid-container system into said first cavity; generating a second pressure configured to deform said first membrane to reduce said first volume or second chamber of said first cavity, thereby pumping said biological fluid to said biological target and/or machine.

Embodiments of the invention generally relate to fluid pumps and fluid pump systems for use in the life sciences field. The pumps may be configured for pumping a biological fluid, such as, but not limited to, blood and fluids used in the manufacturing of proteins, antibodies, biological or complex drugs, vaccines, biopharmaceuticals, tissues, organs etc. These pumps include extracorporeal blood pumps such as those used, e.g., in dialysis machines and heart-lung machines and pumps used in the manufacturing of proteins, antibodies, biological or complex drugs, vaccines, biopharmaceuticals, tissues, organs etc., e.g. using bioreactors.

Embodiments of the invention may overcome one or more problems associated with prior art pumps, such as blood pumps, and/or pump systems such as those described above. The pumps and/or pump systems of the invention may provide a design suitable for use in the life sciences field. The pumps and/or pump systems of the invention may be economical to manufacture and/or may be readily adaptable to a plurality of dimensional characteristics.

Embodiments of the invention may provide a controlled pressure, which may be achieved by a fluid reservoir with a pressure-retaining function. The controlled pressure may be achieved by a suction principle according to some embodiments of the invention.

According to some embodiments, a pump and/or pressure-retaining device may include an upper and lower cavity, where the upper cavity has a pumping function and the lower cavity has a pressure maintenance function and/or pressure retaining function. Through suction the upper cavity may take in fluid (e.g., blood, a fluid used in manufacturing of proteins, biological drugs, vaccines, biopharmaceuticals, etc.). Transport of the fluid between the upper cavity and the lower cavity may include the use of gravity. The lower cavity may deliver fluid with a controlled pressure to the target. The function may be achieved in that every cavity is divided into two chambers that are separated by a membrane. On one side of the membrane is the fluid that is to be pumped with a controlled pressure and on the other side of the membrane is gas pressure/vacuum that exerts a pressure on the membrane and thereby also on the fluid. Check valves may control the direction of transport of the fluid.

Embodiments of the invention may provide an apparatus for pumping a fluid, such as, but not limited to, blood and fluids used in the manufacturing of proteins, antibodies, biological or complex drugs, vaccines, biopharmaceuticals, tissues, organs etc., wherein at least most of the disadvantages in the prior art are avoided. Embodiments of the invention may be configured to fulfil at least one or more of the following advantages:
provide a controlled pressure, such as even/constant pressure or pulsatile pressure;
feed a fluid, such as blood, without any contact with air in order to avoid any contamination from air and/or to avoid the fluid from becoming saturated with air;
provide a pump that does not cause cavitation;
provide a pump presenting very low shear forces;
provide a pump that allows for oxygenation of a fluid, such as blood; and any combination thereof;
provide a pump that has very small degree of material wear and thus release a very small degree of material particles into the fluid stream and risk e.g. particulate embolism;
provide a pump representing high chemical resistance.

In some embodiments, a pump of the present invention may be used for pumping blood. In some embodiments, a pump of the present invention may be used in other areas of the life sciences field such as, for example, where sensitive fluids need to be pumped, including, but not limited to, in the production of proteins, antibodies, vaccines, biopharmaceuticals, tissues, organs, and/or complex molecules and in drug manufacturing of complex compounds. In some embodiments, a pump of the present invention may be used for pumping media where relative coarse material are suspended in media as there is no narrow passages. In some embodiments, the pump can handle corrosive and strong solvent fluids, non lubricating and abrasive fluids and can run dry without damage.

Embodiments of the invention may provide a pump characterized by the absence of rotating shafts, seals, bearings, or the like. Embodiments of the invention may provide an apparatus for extracorporeal circulation that is configured to produce a significant pulsatile flow while still-maintaining a user-specified average flow rate.

The foregoing and other aspects of the present invention will now be described in more detail including other embodiments described herein.

### BRIEF DESCRIPTION OF THE DRAWINGS

FIG. 1 is a schematic illustration of an embodiment of a first cavity in a membrane pump with longitudinal fluid flow according to the invention.

FIG. 2 is a schematic illustration of another embodiment of a first cavity in a membrane pump with longitudinal fluid flow.

FIG. 3 is a schematic illustration of an embodiment of a first cavity in a membrane pump with transverse fluid flow.

FIG. 4 shows a fluid reservoir with longitudinal fluid flow according to embodiments of the invention that includes a first cavity of FIG. 1.

FIG. 5 shows a fluid reservoir with longitudinal fluid flow according to other embodiments of the invention that includes a first cavity of FIG. 2.

FIG. 6 shows a fluid reservoir with longitudinal fluid flow according to still other embodiments of the invention that includes a cavity of FIG. 1 with its chambers reversed.

FIG. 7 shows a fluid reservoir with longitudinal fluid flow according to yet other embodiments the invention that includes a cavity of FIG. 1 with a draining tube.

FIG. 8 shows a fluid reservoir with transverse fluid flow according to embodiments the invention that includes a first cavity of FIG. 3.

FIG. 9 shows a pump system according to embodiments of the invention.

### DETAILED DESCRIPTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, the term “controlled pressure” refers to maintaining the pressure level within the relevant requirements. The pressure level may vary for controlled pressure, but the variance may be maintained within the relevant requirements. A pump and/or pump system of the present invention may be configured to provide a controlled pressure. In some embodiments, the pump and/or pump system is configured to provide a controlled pressure during its use and/or operation. Providing a controlled pressure may avoid a pressure drop or a decrease in pressure when alternating or switching the supply of a fluid from one cavity to another cavity. “Biological fluid” as used herein refers to any fluid, preferably liquid, which may be used in a biological application such as, but not limited to, a liquid used to prepare a biological component, a liquid used in medical devices including experimental and/or training devices (e.g., liquids used in manikins for medical education), and/or a liquid that may be delivered to a target. A biological fluid may comprise blood, a blood analogue fluid, a blood component (e.g., blood serum), amniotic fluid, cerebrospinal fluid, interstitial fluid, lymph, pericardial fluid, water, saline, a fluid comprising a protein, a hormone, an enzyme, an antibody, a biological or complex drug, a vaccine, a therapeutic agent, a biopharmaceutical and/or a starting material and/or intermediate for production of a protein, a hormone, an enzyme, an antibody, a biological or complex drug, a vaccine, a therapeutic agent, a biopharmaceutical, a detergent, a cell, cell culture medium, an Agar agar solution or liquid, an agarol solution or liquid and any combination thereof. In some embodiments, the biological fluid may be blood, a blood analog fluid, a blood component, and any combination thereof. In certain embodiments, a biological fluid may comprise a fluid used in the life sciences where there is a need for sensitive pumping, such as, for example, in the production of various substances and compounds, such as proteins, antibodies, vaccines, biopharmaceuticals, and/or biological or complex molecules and in drug manufacturing of complex compounds. Biological fluid used in bioreactors in tissue engineering include “detergents” during decellularization and oxygenated media, culture media, growth factors and fluids containing cells during recellularization.

The terms “liquid” and “fluid” are used synonymously herein and are intended to refer to any biological fluid.

“Target” as used herein refers to a subject, an organ, a tissue, and/or a machine and/or a laboratory consumable and/or a device for use with a biological fluid. In some embodiments the target is a machine and/or device for use in the life sciences, such as, but not limited to, a machine and/or device for preparing a biological component and/or a medical machine and/or device including experimental and/or training devices (e.g., manikins for medical education). In some embodiments, the target is a biological target. A “biological target” as used herein refers to a subject, an organ, and/or a tissue, and includes an in vitro and/or ex vivo organ and/or tissue. The present invention finds use in both veterinary and medical applications. Subjects suitable to be treated with a method of the invention include, but are not limited to, avian and mammalian subjects. Mammals of the present invention include, but are not limited to, canines, felines, bovines, caprines, equines, ovines, porcines, rodents (e.g. rats and mice), lagomorphs, primates (e.g. simians), non-human primates (e.g. monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult, etc.) may be treated according to the present invention. In some embodiments, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments, the subject is a human adolescent and/or adult. Illustrative avians according to the present invention include chickens, ducks, turkeys, geese, quail, pheasant, ratites (e.g., ostrich) and domesticated birds (e.g., parrots and canaries), and birds in ovo.

In some embodiments, a method of the present invention may be carried out on an animal subject, particularly a mammalian subject such as mice, rats, dogs, cats, livestock and horses for veterinary purposes, and/or for drug preparation, screening and/or development purposes.

According to some embodiments of the present invention, a pump and/or pump system such as that shown in PCT Application No. PCT/SE2012/051175 (WO 2013/062480), filed Oct. 29, 2012 may be suitable for use in the systems described herein; this document is hereby incorporated herein by reference in its entirety.

In many environments it is necessary or desirable to augment or supplant the natural circulation of blood throughout the body by a mechanical means. While many such devices are available, there are still subsisting drawbacks connected with their use. One difficulty is the trauma to which the blood is subjected by mechanical handling, particularly in certain types of pumps. Another difficulty is the danger of air access to the blood being pumped. An invention that would greatly reduce or virtually eliminate trauma to the blood and access of air would be of great interest in the field.

Embodiments of the invention may provide a blood pumping system in which the former disadvantages are overcome. In some embodiments, a fluid pump system of the invention may eliminate polymeric materials such as rubber pumping materials that are prone to eventual thrombogenesis, clotting, calcification, etc.; reduce blood shear stresses to eliminate or reduce blood damage and hemolysis; eliminate or reduce all mechanical contact and wear; reduce the formation of thromboembolic; prevent access of air to the blood; and/or any combination thereof. These and other advantages may be met by embodiments of the present invention.

Embodiments of the invention may provide an arrangement in which the rate of operation of the fluid pump system is carefully and closely coordinated with a desired or natural program. Such a program may yield a pulsatile flow, e.g. mimicking the normal blood flow in the body, but also constant flow rates are possible with the invention. Furthermore, embodiments of the invention may provide a method and a pump for providing pulsatile flow in a return line of an extracorporeal circuit.

Extracorporeal circulation systems are generally used to maintain a subject's (e.g. human or other animal) blood circulation and/or lung function outside the body, e.g. heart-lung machines and extracorporeal membrane oxygenation (ECMO) machines. Generally, a heart-lung machine is used, e.g. in open heart surgery to support the body during the surgical procedure while the heart is stopped. The heart-lung machine is often referred to as the “pump”, and does the work of the heart and lungs during the operation. Generally, the heart-lung machine comprises some form of fluid reservoir, e.g. the fluid reservoir of the current invention that receives the blood from the body, which is normally the responsibility of the heart's right atrium. This blood is then pumped by the machine through, e.g. an oxygenator, a function normally the responsibility of the right ventricle. The oxygenator removes the CO₂ and adds oxygen, which is normally the work of the lungs. The pump then pumps this newly oxygenated blood back to the body, which is normally the work of the left atrium and ventricle of the heart. The heart-lung machine is generally connected to the patient by a series of tubes, e.g. those that a surgical team place. At the end of an operation, the surgeon gradually allows the patient's heart to resume its normal function, and the heart-lung machine is “weaned off”.

Extracorporeal blood circulation systems are also used, for example, for circulating a medical patient's blood through, for example, an artificial kidney. Such a system comprises a tube for withdrawing blood from the patient to the artificial kidney, the artificial kidney itself, and a tube for returning the blood from the artificial kidney to the patient. One or more pumps are used in the system to augment the heart of the patient in causing circulation of the blood through the system. Such systems include various other components, one of which is to assure that the blood returned to the patient does not include any air bubbles.

Embodiments of the invention may prevent and/or reduce the introduction of air into the blood stream and/or eliminate or reduce the danger of damage to the blood corpuscles, trauma or hemolysis. Embodiments of the invention may be configured to provide a pump for use in the extracorporeal circulation of blood, wherein the pump is configured to reduce the introduction of air into the blood stream and/or reduce the danger of damage to the blood corpuscles, trauma or hemolysis compared to other pumps for use in the extracorporeal circulation of blood. Embodiments of the invention may remove, prevent, and/or reduce air that has been incorporated into the blood stream during the use of a pump and/or pump system of the present invention.

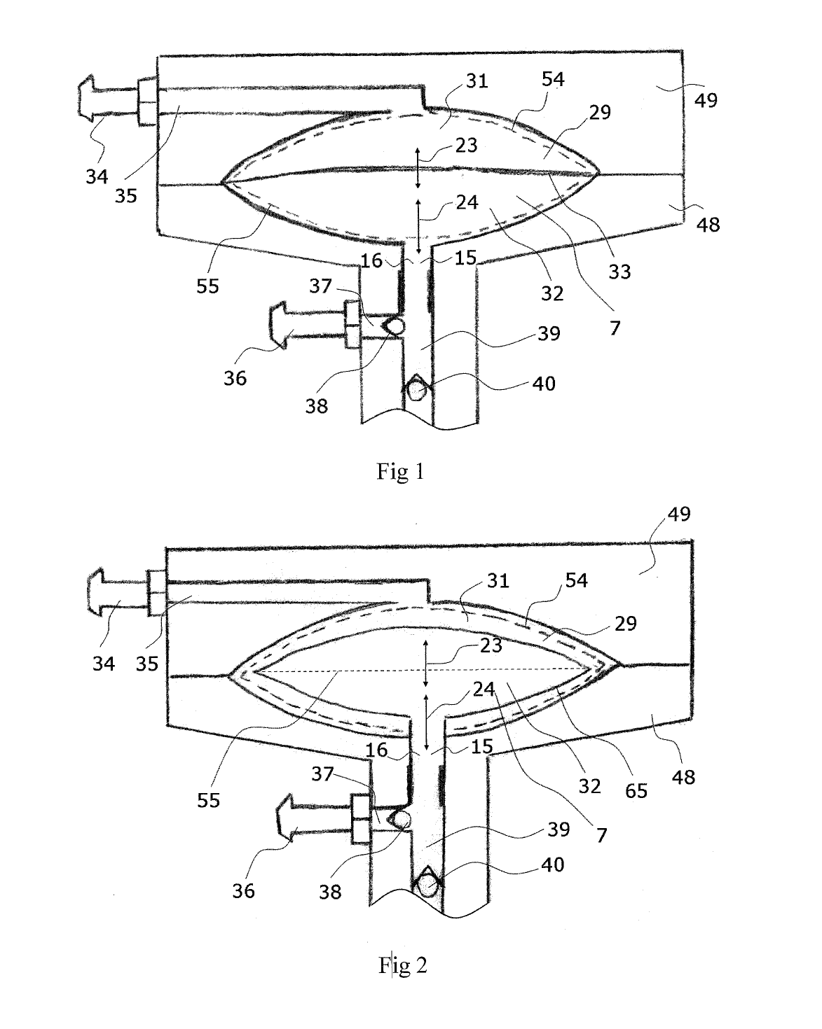

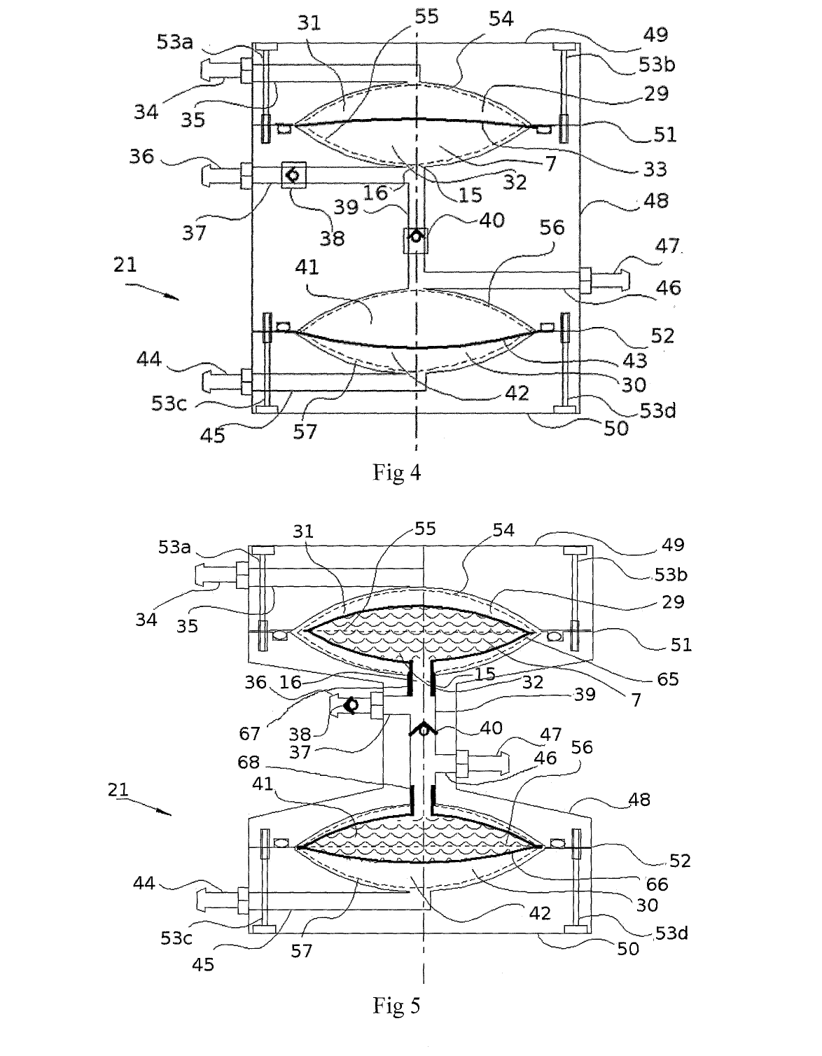

Embodiments of the invention may be used in connection with production of selected substances and compounds, as mentioned in the foregoing. The fluid pump system of the embodiments can thereby be used to pump fluids comprising starting material for the production, pump fluids comprising intermediates obtained in the production, and/or pump fluids comprising the product obtained in the production. Thus, the fluid pump system of the embodiments can further comprise or be connected to bioreactors for the production of selected substances and compounds. Embodiments of the invention may also, or alternatively, be used in connection with preparation of organs or tissues, e.g. during decellularization and/or during recellularization (using e.g. stem cells or other cells) of the organs or tissues. In some embodiments of the invention, the biological fluid is pumped in e.g. a bioreactor or other container. In some embodiments, the biological fluid is pumped in e.g. a bioreactor or other container in a pulsatile flow. In some embodiments, this pulsatile flow can mimic the pulsations in normal blood flow. In some embodiments, the pulsatile flow is used in culturing of cells in vitro or ex vivo. The invention is explained in more detail below with reference to the embodiments illustrated in the drawings. FIG. 1 shows part of fluid reservoir 21 according to an embodiment of the invention, which is further described in FIG. 4 wherein the number references are defined. FIG. 1 shows an embodiment of the invention with fluid 7 and the direction of fluid flow 24 is longitudinal to the moving direction of a membrane 23. A fluid flow inlet 15 and an outlet 16 is located primarily at the peak of the expanding and contracting displacement volume.

FIG. 2 shows part of fluid reservoir 21 according to an embodiment of the invention which is further described in FIGS. 4 and 5 where the number references are defined. FIG. 2 shows an embodiment of the invention where the direction of fluid flow 24 is longitudinal to the moving direction of a membrane 23, where the membrane is in the faint of a bag. A bag may include, but is not limited to, a tube-like membrane and/or bag. A fluid flow inlet 15 and an outlet 16 is located primarily at the peak of the expanding and contracting displacement volume.

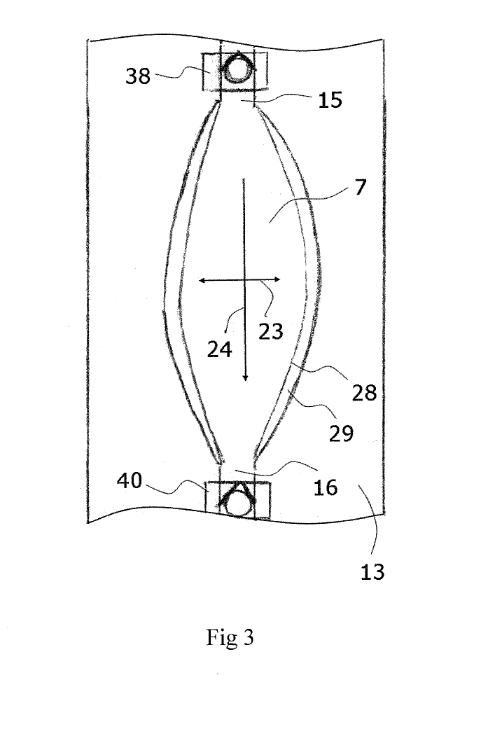

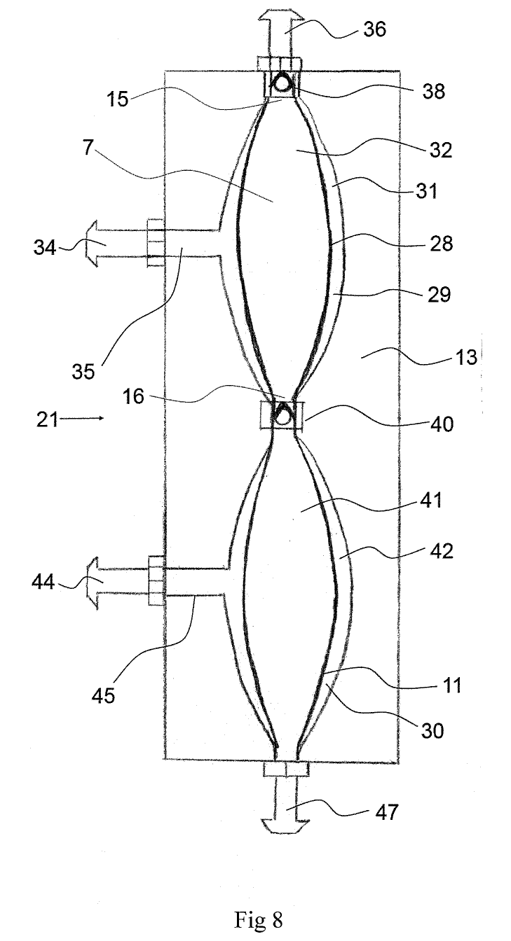

FIG. 3 shows part of fluid reservoir 21 according to an embodiment of the invention which is further described in FIGS. 5 and 8 where the number references are defined. FIG. 3 shows an embodiment of the invention where the direction of fluid flow 24 is transverse to the moving direction of a membrane 23, where the membrane is in the form of a tube-like bag 28. A fluid flow inlet 15 and fluid flow outlet 16 are here located primarily at the nodes of the expanding and contracting displacement volume.

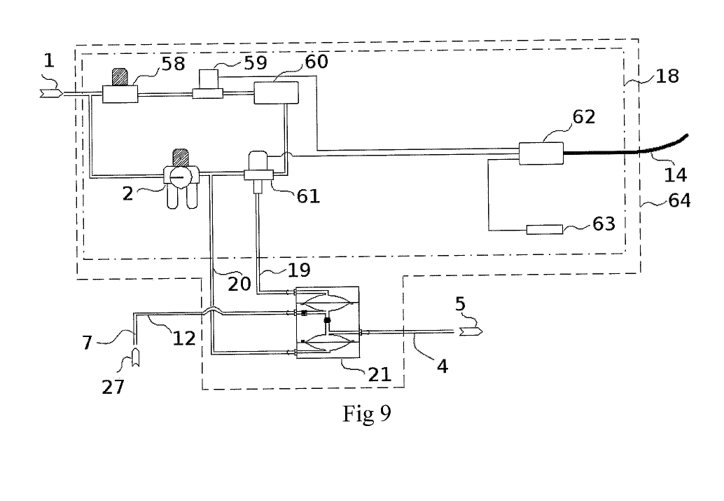

FIG. 4 shows a fluid reservoir 21 according to an embodiment of the invention with reference also to FIG. 9 where some number references are further defined. The fluid reservoir comprises a first cavity 29 and a second cavity 30, the first cavity 29 being located gravitationally above the second cavity 30. The first cavity 29 is defined by a first end 49 which is joined by a first joint 53a, 53b to a central portion 48, and there between a first sealing surface 51. The second cavity 30 is defined by a second end 50 which is joined by a second joint 53c, 53d to the central portion 48, and there between a second sealing surface 52. The first cavity 29 is divided by a first membrane 33 to form a first chamber 31 and a second chamber 32. The first chamber 31 is connected to the control unit 18 via a first conduit 35 and a first connection 34. The second chamber 32 is connected to a first tube 12 via a second conduit 37 comprising a first check valve 38 and an inlet port 36. The second cavity 30 is divided by a second membrane 43 to form a third chamber 41 and a fourth chamber 42. The third chamber 41 is connected to the second chamber 32 via a third conduit 39 comprising a second check valve 40. The third chamber 41 is connected via a fourth conduit 46 to an outlet port 47 to which a target 5 is connected. The fourth chamber 42 is connected via a fifth conduit 45 and a second connection 44 to the media source 1 via the control unit 18 and the fourth tube 20. The central portion 48 and the ends 49, 50 are joined by a first and a second joint 53a-d, here shown as screws threaded into the central portion 48. The joints 53a-d may also be throughbores so that, for example, a pair of opposing joints 53b, 53d are replaced by a screw. They may also be fewer or more than four. The first membrane 33 can move between a first upper position 54 (i.e., a first deformed position) and a first lower position 55 (i.e., a second deformed position) adjacent to the upper and lower convex walls, respectively, of the first cavity 29. The second membrane 43 can move between the second upper position 56 (i.e., a third deformed position) and the second lower position 57 (i.e., a fourth deformed position) adjacent to the upper and lower convex walls, respectively, of the second cavity 30. A fluid flow inlet 15 and an outlet 16 is located at the bottom of the second chamber 32. It is to be understood that the way in which the connections are oriented and arranged to the fluid reservoir 21 is in no way to be construed as a limitation of the scope of protection. The seal of the sealing surfaces 51, 52 in certain embodiments may be accomplished by means of an O-ring, or by both or one of the membranes 33, 43 being extended so as to also cover this area.

FIG. 5 shows a further embodiment of a fluid reservoir 21 of a pump system according to the invention. The main differences from FIG. 4 are that the first membrane 33 has been replaced by a first bag 65 and the second membrane 43 has been replaced by a second bag 66. A first chamber 31 is formed in the space outside the first bag 65 in the first cavity 29. A second chamber 32 is formed in the space inside the first bag 65. A third chamber 41 is formed in the space inside the second bag 66. A fourth chamber 42 is formed in the space outside the bag in the second cavity 30. The first check valve 38 can be integrated in the inlet port 36 on the outside of the central portion 48. It can also be integrated in the first tube 12, which is connected to the inlet port 36. The bags 65, 66 have spigots 67, 68, which are connected to the central portion 48. In order to save material, the central portion 48 has been shaped as a tapered waist, but other shapes or designs may also be used.

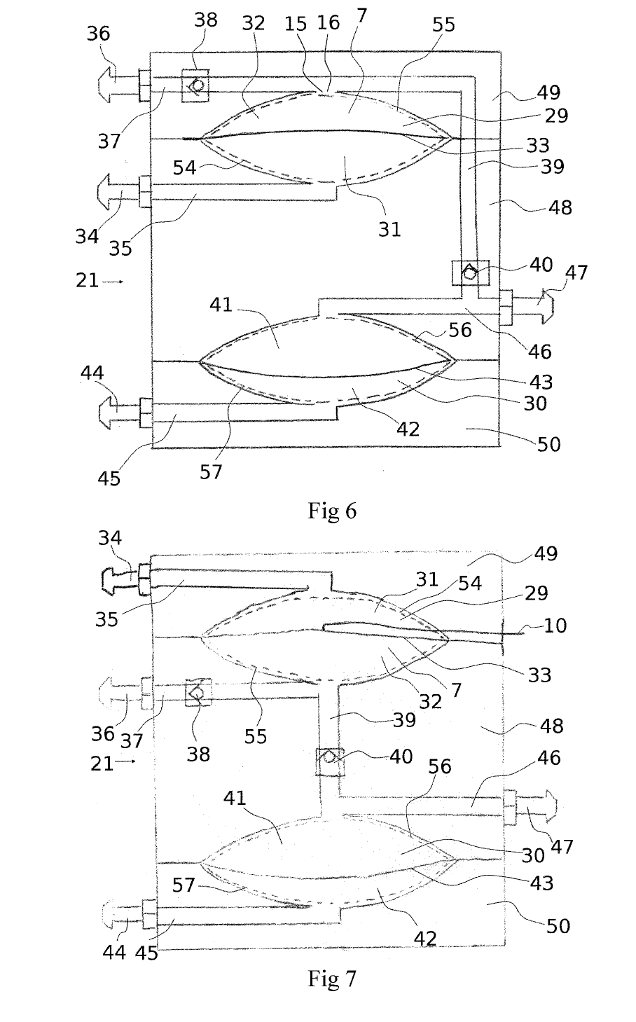

FIG. 6 shows another embodiment of a fluid reservoir 21 of a pump system according to the invention. The main differences from FIG. 4 are that the position of first chamber 31 and the second chamber 32 is reversed, i.e. the second chamber 32 is above the first chamber 31. Thus, the fluid flow inlet 15 and the outlet 16 are located at the top of the second chamber 32. This involves different routing and/or placement of at least the third conduit 39 and the second conduit 37, the first check valve 38, the inlet port 36 and the first conduit 35, the first connection 34 compared to the fluid reservoir 21 in FIG. 4.

FIG. 7 shows another embodiment of a fluid reservoir 21 of a pump system according to the invention. The main difference from FIG. 4 is that there is a draining tube 10 connected to the first membrane 33.

FIG. 8 shows a further embodiment of a fluid reservoir 21 of a pump system according to the invention with transverse fluid flow. The main differences from FIG. 5 are that the first bag 65 has been replaced by a first tube-like bag 28 and the second bag 66 has been replaced by a second tube-like bag 11. At least the first end 49, the central portion 48 and the second end 50 is replaced by the housing 13.

FIG. 9 shows a pump system 64 according to an embodiment of the invention. The system comprises a fluid reservoir 21, a control unit 18, a fluid-container system 27 and a target 5. The control unit 18 is connected to the fluid reservoir 21 via a third tube 19 and a fourth tube 20. The fluid-container system 27 is connected to the fluid reservoir 21 via a first tube 12 and the target 5 which is connected to the fluid reservoir 21 via a second tube 4. The control system 18 comprises a pressurization system 1, 2, 58, 59, 60, 61. The pressurization system 1, 2, 58, 59, 60, 61 may be used to regulate the pressure in a first cavity 29 and/or a second cavity 30 in a fluid reservoir 21, and may apply a positive or negative pressure to the first cavity 29 and/or second cavity 30. The pressurization system 1, 2, 58, 59, 60, 61 may include a vacuum system 58, 59, 60. The vacuum system 58, 59, 60 comprises a first regulator 58, a first solenoid valve 59 and a vacuum injector 60. Compressed air for the vacuum system 58, 59, 60 may be obtained from an external compressed-air source 1, typically about 4 to about 10 bar or any range and/or individual value therein. In some embodiments, the compressed air for the vacuum system 58, 59, 60 may be in a range of 4 to 10 bar or any range and/or individual value therein. The external compressed-air source 1 supplies the regulator 58, which down-regulates the pressure to normally about 1 to about 4 bar or any range and/or individual value therein. In some embodiments, the pressure is down-regulated to 1 to 4 bar or any range and/or individual value therein. The regulator 58 feeds the air connection of the vacuum injector 60 via the first solenoid valve 59, which controls the vacuum injector 60. The vacuum connection of the vacuum injector 60 is connected to a second solenoid valve 61. The external compressed-air source 1 also supplies a second regulator 2 in the control system 18. The second regulator 2 down-regulates the pressure of supplied compressed air to the desired pressure of the fluid, normally about 60 to about 130 mmHg or any range and/or individual value therein. In some embodiments, this desired pressure is in a range of 60 to 130 mmHg. The second regulator 2 is connected to the fourth tube 20 and the second solenoid valve 61. The second solenoid valve 61 controls the pressure in the third tube 19 using vacuum from the vacuum ejector 60 or pressure from the second regulator 2. A controller 62 connected to a power voltage source 14 controls the two solenoid valves 59, 61 which are operated simultaneously. Connected to the control 62 may be a photocell 63 or other activation means. The fluid reservoir 21 draws fluid 7, such as, but not limited to, blood, from the fluid-container system 27 via a first tube 12 via the inlet port 36 and the via the outlet port 47 to a second tube 4 feeding fluid with a controlled pressure to a target 5 via the outlet 47.

The function of the fluid reservoir 21 and a pump system 64 according to an embodiment of the invention will now be described with reference to FIG. 4 and FIG. 9. The fluid reservoir 21 comprises two interconnected cavities 29, 30, a first cavity 29 and second cavity 30, the first cavity 29 being located gravitationally above the second cavity 30. The first cavity 29 has a suction function and the second cavity 30 has a pressure-retaining function. Feeding from the first cavity 29 to the second cavity 30 may comprise the use of gravity. In this way, a supply of a fluid, such as blood, may be obtained at the correct pressure level and with very small pressure fluctuations to a target 5; thus a controlled pressure may be achieved. This function will now be described in detail.

The second solenoid valve 61 is provided with pressure from the second regulator 2 and vacuum from the vacuum ejector 60. The third tube 19 is switched between pressure and vacuum by the second solenoid valve 61 at a switching rate of about 0.01 to about 10 times per minute or any range and/or individual value therein. In some embodiments, the switching rate is about once per minute. The first chamber 31 is in communication with the second solenoid valve 61 via the first connection 34, the first conduit 35 and the third tube 19. When the first chamber 31 is in the vacuum phase, the first membrane 33 moves upwards. A pressure below ambient pressure develops in the second chamber 32, causing the second check valve 40 in the third conduit 39 to close and the first check valve 38 to open, and fluid 7 is drawn from the inlet port 36 connected to the fluid-container system 27, causing the second chamber 32 to fill with the fluid 7. After the set time value in the controller 62 has been reached, the first membrane 33 has come close to or reached its first upper position 54 (i.e., a first deformed position), whereupon the second solenoid valve 61 switches to the pressure phase. Thus, a pressure develops in the first chamber 31 and the first check valve 38 closes. The same pressure that is fed to the first chamber 31 via the third tube 19 is also fed to the fourth chamber 42 via the fourth tube 20. In some embodiments, the pressure in the first chamber 31 and the fourth chamber 42 are substantially constant. “Substantially constant” as used herein refers a measureable value, such as pressure, on average, varying less than about 20%, 15%, 10%, 5%, 1% or less over a period of time. Because the second chamber 32 is gravitationally located above the third chamber 41, gravity creates a pressure difference between the second chamber 32 and the third chamber 41, causing the second check valve 40 to open and the fluid 7 to flow downwards to fill the third chamber 41. After the set time value in the controller 62 has been reached, the first membrane 33 has approached or reached its first lower position 55 (i.e., a second deformed position) and the second membrane 43 has approached or reached its second lower position 57 (i.e., a fourth deformed position), whereupon the solenoid valve 61 switches over to the vacuum phase, and the entire cycle is repeated. The fourth chamber 42 is under constant pressure from the second regulator 2, causing the fluid 7 to be fed with a controlled pressure to the outlet port 47 via the fourth conduit 46 and the second tube 4 to a target 5.

According to some embodiments, a fluid reservoir 21 and a pump system 64 of the present invention may be suitable to supply pulsatile pressure to the outlet port 47 and further to the target 5. A pulsatile pressure may be achieved by controlling the second regulator 2 to supply the desired pulsatile pressure. As the pressure is synchronized in time and level on the first cavity (29) and the second cavity 30 the gravity principle may work even during pulsatile pressures.

The pressure may, for example, be set to switch between two levels to e.g. mimic (as in) the human body systolic blood pressure and diastolic blood pressure. In some embodiments, the pressure may be configured to switch between a systolic blood pressure, which may be in a range of about 80 to about 130 mmHg or any range and/or individual value therein, such as, in some embodiments about 90 to about 130 mmHg (e.g. 90 to 130 mmHg), and in further embodiments about 90 to about 120 mmHg (e.g. 90 to 120 mmHg), and a diastolic blood pressure, which may be in a range of about 50 to about 90 mmHg (e.g. 50 to 90 mmHg) or any range and/or individual value therein, such as, in some embodiments about 60 to about 90 mmHg (e.g. 60 to 90 mmHg), and in further embodiments about 60 to about 80 mmHg (e.g. 60 to 80 mmHg). In some embodiments, e.g. for the application in bioreactors, such as in connection with production of selected compounds and substances, the pressure may be in the range of 2 to about 35 mmHg and up to 130 mmHg. According to some embodiments, a fluid reservoir 21 and a pump system 64 of the present invention may be configured to supply pulsatile pressure to the inlet port 36. To achieve a pulsatile pressure, the vacuum system 58, 59, 60 may be configured to supply the desired pulsatile pressure.

In some embodiments, the pump system 64 includes one or more flow meters and/or pressure sensors (not shown). The flow meters and/or pressure sensors may be configured to provide information to the controller 62 to dynamically alter the operation of the pump. In certain embodiments, the flow meters and/or pressure sensors may be configured to provide information to the controller 62 to provide a pulsatile pressure. In some embodiments, a pressure sensor may be used to detect an increase and/or decrease in pressure and/or may be configured to deactivate the pump such as, for example, if there is a fluid leak.

A pump and/or pump system according to embodiments of the invention may be safer for a target and/or biological fluid compared to a conventionally used pump, such as a roller pump. For example, in some embodiments, a pump and/or pump system may have a small or negligible increase (e.g., an increase at about 10% or less, such as 5% or less) in pressure if a blockage occurs in the pump and/or pump system. Blockages in conventionally used pumps, such as roller pumps may result in high pressures and/or high vacuum levels, which can damage the blood and/or otherwise be hazardous to the target.

A pump and/or pump system according to embodiments of the invention may be advantageous in applications where there is a high demand on constant fluid flow and/or pressure for a target compared to a conventionally used pump in these applications, such as a syringe pump.

In some embodiments, a pump and/or pump system according to embodiments of the invention may have the same and/or improved pressure stability than conventionally used pumps in these applications, such as a syringe pump, but the pump and/or pump system may not have the disadvantages associated with refilling and thus may supply fluid continuously.

A pump and/or pump system according to embodiments of the invention may be advantageous to pumping heat sensitive fluids compared to conventionally used pumps which can generate heat by friction between the moving parts and/or from motor components. In some embodiments, a pump and/or pump system according to embodiments of the invention may have a low or negligible heat formation.

A pump and/or pump system according to embodiments of the present invention may always have a positive and controlled pressure. A pump and/or pump system of the present invention may not have intermittent pressure spikes like many conventionally used pumps.

The positioning of the fluid reservoir 21 may have different “schemes” in relation to the other parts, such as the oxygenator in a cycle of a medical system, for example in a dialysis system or heart lung machine and that all such positions are included in the invention.

The sealing surfaces 51, 52 may comprise O-rings and/or gaskets. In some embodiments, the sealing surfaces 51, 52 comprise grooves to receive the O-rings and/or gaskets. The membranes 33, 43 may be extended to cover the sealing surfaces 51, 52. Other means of sealing for the sealing surfaces 51, 52 and thus to seal the first and second cavities 29, 30 and chambers 31, 32, 41, 42 may be used, including, but not limited to, adhesives, heat bonding, and/or welding. In certain embodiments, the membranes 33, 43 may be fabricated with similar materials that will bond together when heated.

The material for elements of the pump system 64, such as those in contact with the pumped liquid (e.g., tubes, membranes, check valves, connectors and cavity walls) may be selected based on the biological fluid that may come into contact with one or more of the elements. For example, when the biological fluid is blood, the material's tendency to induce clot formation may be considered. In some embodiments, one or more elements of the pump system 64 comprise a biocompatible material (i.e., a material suitable for use with a biological fluid and/or compatible with a target) and/or comprise a surface treated with biocompatible material. Exemplary biocompatible materials include, but are not limited to, titanium; solid pyrolytic carbon such as Biolite™ carbon surface; Nitinol, which is formed by alloying nickel and titanium and stainless steel; biocomposite coatings containing cells; protein coatings; hemocompatible materials and/or coatings; and any combination thereof. Elements of a pump and/or pump system may comprise non-biocompatible materials, biocompatible materials such as biocompatible metals, and any combination thereof.

In certain embodiments, a pump of the present invention, such as, but not limited to, a blood pump or a pump for production of e.g. proteins, antibodies, biological drugs, vaccines, complex drugs, biopharmaceuticals or tissues/organs, may be configured such that one or more of the surfaces of the pump which interface with the fluid (e.g. blood) do not induce biological changes in the fluid elements. For example, for blood, at least one surface of a pump may be configured to not be thrombogenic. A pump of the present invention may be configured such that the fluid dynamics of the pump are designed to avoid turbulence, shear, cavitation, stagnation, flow separation and the like that may cause mechanical damage.

In some embodiments, a pump of the present invention, such as, but not limited to, a blood pump, may be sterile and/or free of toxic materials when put into use and may optionally remain so as long as it is in use. In certain embodiments, the risk for bacterial growth on structures in the pump may be reduced.

In a pump system according to some embodiments of the invention, all electricity is kept separate from the fluid. In the exemplary embodiments, all electrical components have been concentrated to the control unit 18, which can be installed outside the controlled environment.

According to some embodiments, a pump system 64 may be configured to provide low shear forces and/or may reduce or eliminate cavitation, which may be explained by the fact that the fluid reservoir 21 operates at low vacuum levels. Because the fluid reservoir 21 has a large area in the first membrane 33 and a great displacement in the second chamber 32, sufficient fluid flow may be achieved using a low pump frequency, which means that a low vacuum may be used. The suction force may be determined by the vacuum level in the first chamber 31. The vacuum level may be adjusted by the regulator 58.

If gas bubbles enter into the second chamber 32, the gas bubbles will be accumulated in the top of the same second chamber 32. These gas bubbles may give the pump less pumping efficiency. By turning the fluid reservoir 21 upside down, the air enters the third chamber 41 via the second check valve 40. When turning back the fluid reservoir 21 to its original orientation, the gas bubbles follow the flow of liquid to the outlet port 47 via the fourth conduit 46 and the pump has been fully liquid filled and is thereby degassed and ready to use.

The embodiments of fluid reservoir 21 in FIGS. 6 and 7 may avoid the need of turning of the fluid reservoir 21 upside down to remove existing gas bubbles. In FIG. 6 the gas bubbles are entering and leaving the first chamber 32 thru the fluid flow inlet 15 and outlet 16, which is located at the top of same second chamber 32. A degassing manoeuvre can be made in several ways. An example is by first filling the second chamber 32 with fluid so that the first membrane 33 will approach or reach the first upper position 54 (i.e., a first deformed position). By pressurizing the fourth chamber 42 the second membrane 43 will approach or reach the second upper position 56. By now pressurizing the first chamber 31 and releasing the pressure in the fourth chamber 42 the fluid and the gas bubbles will be forced through the second check valve 40 and into the third chamber 41. By pressuring the fourth chamber 42 again the gas bubbles will follow the flow of liquid to the outlet port 47 via the fourth conduit 46 and the pump has been fully liquid filled and is thereby degassed.

In FIG. 7 there is a draining tube 10 connected to the first membrane 33. The function of the draining tube 10 is to evacuate gas bubbles in the fluid which are accumulated in the top of the second chamber 32.

In some embodiments, such as those in FIGS. 1, 2, 4, 5, 6 and 7, there could potentially be a risk of stagnant zones in the second chamber 32 and the third chamber 41 where fluid, such as, but not limited to, blood, may coagulate. The embodiment described in FIG. 8 may be configured to avoid these stagnant zones and/or to avoid the need of turning of the fluid reservoir 21 upside down to remove existing gas bubbles. As can be seen in FIG. 8, the direction of liquid flow 24 is transverse to the moving direction of the membrane 23 and no dead ends and stagnant zones are present as there is flow in the whole compartment of the first tube-like bag 28 and the second tube-like bag 11.

It is understood that any non-solid medium may be used for the pressurization and vacuumization of the first chamber 31, and for the pressurization of the fourth chamber 42, such as a liquid.

In certain embodiments, the orientation of the cavities is such that the first cavity 29 is located gravitationally above the second cavity 30. However, as those skilled in the art will recognize, the invention is not limited to this orientation. The pump may work with other orientations of the cavities 29, 30. For example, the cavities 29, 30 may be spaced apart and the central portion 48 comprising the third conduit 39 and the second check valve 40 may be replaced by a pipe connecting the cavities. Also, the first cavity 29 need not be located at a gravitationally higher level than the second cavity 30. Location of the cavities side-by-side on the same level and connected at the bottom via the third conduit 39 may also be a suitable arrangement.

The first check valve 38 may be integrated in the inlet port 36 on the outside of the central portion 48. It may also be integrated in the first tube 12, which is connected to the inlet port 36. In some embodiments, the second check valve 40 may have a very low opening force and/or a low flow resistance to reach the desired fluid flow from the second chamber 32 to the third chamber 41, which may be accomplished using gravity. The check valve design may be made in the form a rubber wing, ball and seat or check valves similar to the vein valves or heart valves in the human body. These design variants are known to a person skilled in the art of check valves.

In certain embodiments, the first check valve 38 may have such a low flow resistance that the desired fluid flow is reached and still within acceptable shearing forces. The check valves may have a different design than what is mentioned if they meet the same functional requirements.

According to some embodiments, an alternative to the draining tube 10 in FIG. 7 is to have a draining hole in the wall of the second chamber 32, i.e. in the lower convex wall of the central portion 48 defining one of the sides to the first cavity 29. In some embodiments, this draining hole may be positioned at the top region of the second chamber 32 to effectively evacuate gas bubbles which may be in the fluid.

In FIG. 8, the shape of the first tube-like bag 28 and/or the second tube-like bag 11 may be selected from the shape of a tube, oval or round or hybrids thereof. The bag may be thin walled to have negligible self-resistance in terms of its movement in the cavities. In some embodiments, the shape is tube-like as this may provide less stagnant zones.

In some embodiments, the invention may be used in the production of proteins, antibodies, biological drugs, complex drugs, vaccines, biopharmaceuticals, and/or the like, and any combination thereof. In some embodiments, one or more parts of a pump and/or pump system of the present invention that are in contact with the fluid may be single-use. This may, for example, reduce cross-contamination between batches and/or different products. This may mean that no cleaning of the pump and/or pump system may be necessary when switching between different batches and/or products. This single-use embodiment may also be used for other embodiments of the invention where, for example, contamination may be a problem, e.g. in blood pumps and extracorporeal blood pumps.

Previously described features of the invention, such as, for example, low shear pressure and controlled pressure, may be advantageous in the production of proteins, antibodies, biological drugs, complex drugs, vaccines, biopharmaceuticals, tissues, organs and/or the like since these can be sensitive to e.g. shear and high pressure.

The central portion 48 may not include the first conduit 37, but the first conduit 37 comprising the first check valve 38 may be directly connected to the second chamber 32 in the first cavity 29. Similarly, the second conduit 46 may be directly connected to the third chamber 41 in the second cavity 30.

The choice of materials for, and the design of, the membranes 33, 43 may be made in such a way that they have a negligible self-resistance in terms of their movement in the cavities 29, 30. Otherwise, a non-controlled pressure may result. In some embodiments, the membranes 33, 43 may comprise a thin foil having the same convex shape as the cavities 29, 30. This may allow the membranes 33, 43 to reach their end positions 54, 55, 56, 57 without tensioning. In some embodiments, the membranes 33, 43 present very good mechanical properties in terms of fatigue and/or may be resistant to chemicals. Examples of suitable materials include, but are not limited to plastics such as polypropylene (PP), polyethylene (PE), fluorinated ethylene propylene (FEP), polyvinylchloride (PVC), polyurethane (PU), and any combination thereof.

In certain embodiments, a membrane may comprise a metal foil. In some embodiments, a membrane may comprise a biocompatible material and/or have a surface treated with a biocompatible material. However, other materials presenting suitable properties may also be used. The accuracy of the second regulator 2 and the self-resistance of the membranes 33, 43 may determine how well the pressure in the outlet port 47 is controlled. In some embodiments, the regulator is a so-called precision regulator. When the choices of regulator 2 and membranes 33, 43 are appropriate, a controlled pressure is obtained. The fluid reservoir 21 withstands high pressures and high vacuum levels, because the membranes are not exposed to increased load as a result of the fluid 7 running out or high pressures in the third tube 19 and/or the fourth tube 20, or high vacuum in the third tube 19 because there is a medium on both sides of the membranes 33, 43, whose pressures cancel each other, but if the medium in any of the chambers 31, 32, 41, 42 disappears, a pressure situation may occur where the first membrane 33 reaches its first upper position 54 or its first lower position 55, and/or where the second membrane 43 reaches its second upper position 56 or its second lower position 57. This means that the membranes abut against and are supported by the wall of the convex cavities 29, 30. In order to ensure that the membranes 33, 43 are not exposed to damage, in the regions where the cavities 29, 30 are joined to the first, third and fifth conduits 35, 39, 45, when they have reached their upper or lower positions 54, 55, 56, 57, it is possible, for example, to reinforce the membranes 33, 43 in these very regions. Another option could be for the opening, which is formed where the cavities 29, 30 are connected to the conduits 35, 39, 45, to have some kind of support; for example, a coarse mesh could cover the hole, which would then support the membranes 33, 43 when they have reached their upper and lower positions 54, 55, 56, 57 respectively. The cavities 29, 30 are designed to be sufficiently robust to withstand the pressure that an external industrial compressed-air source 1 can generate.

In certain embodiments, a gas-permeable membrane (i.e., a membrane that is permeable to at least one gas such as oxygen, carbon dioxide and/or nitrogen gas, but consequently not liquid) may be used in the first cavity 29 and/or second cavity 30. The gas-permeable membrane may cause the fluid drawn into the first cavity 29 to be degassed during the vacuum phase and/or may add gas during the pressure phase in the first cavity 29 and/or the pressure in the second cavity 30. It is understood that it is a great advantage to be able to integrate the degassing and/or addition of a gas into the fluid, such as, but not limited to, blood. In some embodiments, the fluid is blood in a feeding operation in an extracorporeal blood pumping system such as e.g. a heart-lung machine. The sizes of the cavities 29, 30 in the fluid reservoir 21 are chosen so that, in relation to the flow to the target 5, the time is sufficient for effective degassing and/or addition of gas. The degree of degassing may be controlled by the first regulator 58, which controls the vacuum level of the vacuum injector 60.

An embodiment completely without membranes is also contemplated. This embodiment is configured to provide careful control of the fluid level in the first cavity 29 to ensure the first cavity 29 is not over-filled and fluid is carried up into the third tube 19 and further up into the control system 18.

Degassing of the fluid, such as, but not limited to, blood, may then be achieved in the first cavity 29 by providing the first conduit 37 with a shut-off valve as a supplement or alternative to the first check valve 38. Then, high vacuum may be allowed without the fluid being drawn further into the third tube 19 and further up into the control system 18.

In an embodiment with laterally arranged cavities according to the principle of communicating vessels, the pressure medium acts on the upper surface of the fluid in both cavities 29, 30, and if an inert gas, such as, but not limited to, nitrogen gas, is used as the pressure medium 1, it is understood that membranes can be dispensed with. Nevertheless, membranes can serve the purpose of protecting the fluid, such as, but not limited to, blood, against other pollutants, such as dust, which may be present in the pressure medium. Within the scope of the invention it is, of course, possible to combine membranes, or to include no membrane or bag in the cavities based on what is suitable for the application. For example, in some embodiments, membrane and bag constitute a combination, and in further embodiments no membrane and membrane constitute another combination, etc.

The check valve 40 in the third conduit 39 is closed during the vacuum phase, and the pressure in the fourth chamber 42 feeds the fluid in the third chamber 41 into the target 5. It is understood that in an embodiment where the second conduit 46 is connected directly to the third chamber 41, i.e., the third conduit 39 and the second conduit 46 are completely separated, the check valve may be disposed in the inlet to the third chamber 41. Similarly, the first check valve 38 could be disposed in a separate inlet for the first conduit 37 to the second chamber 32.

In accordance with the embodiment shown in FIG. 5, it is particularly suitable to arrange the check valve 40 in the inlet spigot 68 of the bag 66.

The cavities 29, 30 may have another shape than that illustrated in FIG. 5. In some embodiments, the cavity shape is convex to support membranes or bags. The membranes 33, 43 and bags 65, 66 may have a different shape or be made of other materials than what is mentioned if they meet the same or similar functional requirements.

The vacuum source can also be another than the above-mentioned vacuum system 58, 59, 60.

Of course the conduits 35, 37, 39, 45, 46 disposed in the fluid reservoir 21 and through which fluid 7 and pressure medium flow, need not be arranged as shown in the drawings. It is understood that these conduits can be designed with different routes through the ends 49, 50 and the central portion 48, as the shape of these parts can be varied as long as they withstand the pressure that can be generated by an external industrial compressed-air source 1 or any range and/or individual value therein. Likewise, it is understood that the shape of the cavities can be varied, making it possible to adapt the encompassing ends 49, 50 and the central portion 48 accordingly. Therefore, the connecting conduits may be routed through these parts differently from what is shown in the drawings.

In a contemplated variant of the control system 18, the second regulator 2 is disposed downstream of the first regulator 58, the benefit being increased protection against high pressure to the fluid reservoir 21.

It is understood that within the scope of the invention, connections other than tubes can be used; for example, it is possible to use pipes. It is further understood that the tubes or pipes may constitute means integrated in the fluid reservoir 21 for connecting peripheral equipment, or that the systems and components connected to the fluid reservoir 21, such as fluid-container systems, targets, control systems, can include these necessary means for interconnection.

According to some embodiments, a method of pumping a biological fluid 7 to a target 5 may be provided. The method may comprise providing a fluid pump system 64. The fluid pump system may comprise a fluid reservoir 21 comprising at least one first cavity 29 and at least one second cavity 30 in fluid communication with each other and configured to accommodate the biological fluid 7, wherein the at least one second cavity 30 is in fluid communication with the target 5. The fluid pump system may further comprise a fluid-container system 27 in fluid communication with the fluid reservoir 21, preferably in fluid communication with the at least one first cavity 29 in the fluid reservoir 21, and a pressurization system 1, 2, 58, 59, 60, 61 configured to generate a pressure in the at least one first cavity 29 and the at least one second cavity 30. The method may comprise generating a first pressure below ambient pressure in the at least one first cavity 29 using the pressurization system 1, 2, 58, 59, 60, 61, thereby causing the biological fluid 7 to flow from the fluid-container system 27 into the at least one first cavity 29. In some embodiments, a negative pressure may be applied to the at least one first cavity 29. The method may further comprise generating a second pressure in the at least one first cavity 29 using the pressurization system 1, 2, 58, 59, 60, 61, thereby causing the biological fluid 7 in the at least one first cavity 29 to flow into the at least one second cavity 30. The second pressure may be applied to the at least one second cavity 30, thereby pumping the biological fluid 7 to the target 5.

In some embodiments, the second pressure may comprise a positive pressure. The second pressure may be constantly applied to the at least one second cavity. According to a method of the present, the fluid 7 in the at least one first cavity 29 may flow by gravity into the at least one second cavity 30. In certain embodiments, the at least one first cavity and the at least one second cavity may have the same second pressure. The at least one second cavity may have the second pressure before the second pressure is applied to the at least one first cavity. The method may be configured to provide a time period during which the second pressure is applied to both the at least one first cavity and the at least one second cavity (i.e., both of the at least one first and second cavities experience the second pressure at the same time). For example, the second pressure may be constantly applied to the at least one second cavity and the second pressure may intermittently be applied to the at least one first cavity. Thus, the method may comprise one or more periods of time in which the second pressure is applied to both the at least first and second cavities. This may allow for the at least one first and second cavities to have the same external gas pressure and the driving force to move the fluid from the at least one first cavity to the at least one second cavity may be the difference in height between the at least one first and second cavities (i.e., a gravity force). The second pressure applied to the at least one first cavity and/or the at least one second cavity may be a pulsatile pressure. In some embodiments, the first pressure applied to the at least one first cavity may be a pulsatile pressure.

According to some embodiments, the fluid level in the at least one first cavity (i.e., a first fluid level) may be higher than the fluid level in the at least one second cavity (i.e., a second fluid level). In this embodiment, when the same pressure is applied to the at least one first and second cavities, the external pressures on the at least one first and second cavities are the same. Since the first fluid level is higher than the second fluid level, the transport of fluid from the at least one first cavity to the at least one second cavity may be driven by the weight of the fluid in the first fluid level (i.e., by gravity).

The at least one first cavity 29 may comprises a first membrane 33, thereby forming a first chamber 31 and a second chamber 32. The least one second cavity 30 may comprise a second membrane 43, thereby forming a third chamber 41 and a fourth chamber 42. In other embodiments, the at least one first cavity 29 may comprise a first bag 65, thereby forming a first chamber 31 and a second chamber 32 and/or the at least one second cavity 30 may comprise a second bag 66, thereby forming a third chamber 41 and a fourth chamber 42. In some embodiments, the first membrane 33 and/or the second membrane 43 and/or the first bag 65 and/or the second bag 66 comprise a polymeric material.

A method of the present invention may comprise adding at least one gas through the first membrane 33 and/or first bag 65 and/or the second membrane 43 and/or second bag 66. In some embodiments, the at least one gas may comprise oxygen. This addition of oxygen may reduce and/or eliminate an oxygenator step in a cycle of a medical system, for example in a dialysis system or heart lung machine.

In some embodiments, the method may comprise the step of detecting a first deformed position of the first membrane 33 and/or first bag 65. The detecting step may occur prior to the step of generating the second pressure in the at least one first cavity 29. In certain embodiments, the detecting step may be carried out using a sensor.

A method of the present invention may be configured to provide a shear force of less than about 15 Pa or any range and/or individual value therein, on the biological fluid 7. In some embodiments, a shear force of less than about 10, 5, 1, 0.5, 10⁻², 10⁻⁴, 10⁻⁶ Pa or less may be provided by a method of the present invention. In certain embodiments, the first pressure and the second pressure may be substantially constant.

Any suitable volume may be pumped according to a method of the present invention. In some embodiments, the amount of the biological fluid 7 pumped to the target 5 may be about 2 to about 55 mL/min or 250 to about 350 mL/min or any range and/or individual value therein. In some embodiments, the amount of the biological fluid 7 pumped to the target 5 may be about 1 mL/min to about 10 L/min or any range and/or individual value therein. In some embodiments, the amount of the biological fluid 7 pumped to the target 5 may be about 1 mL/min to about 400 mL/min. Thus, in general the amount of the biological fluid 7 pumped to the target 5 could be in a range of 2 mL/min up to about 10 L/min.

In some embodiments, the amount of the biological fluid 7 pumped to the target 5 may be about 400 to about 1000 mL/min or any range and/or individual value therein. In some embodiments, the amount of the biological fluid 7 pumped to the target 5 may be 400 to 1000 mL/min or any range and/or individual value therein. In other embodiments, the amount of the biological fluid 7 pumped to the target 5 may be about 5 to about 10 L/min or any range and/or individual value therein. In some embodiments, the amount of the biological fluid 7 pumped to the target 5 may be 5 to 10 L/min or any range and/or individual value therein.

According to some embodiments, a method of the present invention may be configured to provide a pulsatile flow of the biological fluid 7 to the target 5. To achieve the pulsatile flow, a pulsatile pressure may be generated and/or applied according to an embodiment of the present invention.

A pump system 64 for use in a method of the present invention may comprise a blood pump (e.g., an extracorporeal blood pump), a pump for production of proteins, antibodies, biological drugs, complex drugs, vaccines, biopharmaceuticals, tissues, organs, and/or the like, and any combination thereof. The pump system 64 may be connected to a dialysis machine, such as, but not limited to hemodialysis machine, a peritoneal dialysis machine, a hemofiltration machine, a hemodiafiltration machine, or an intestinal dialysis machine.

A further embodiment of the present invention may comprise an extracorporeal blood pump system. The extracorporeal blood pump system may comprise a fluid reservoir 21 comprising at least one first cavity 29 and at least one second cavity 30 in fluid communication with each other and configured to accommodate blood; a fluid-container system 27 in fluid communication with the fluid reservoir 21; and a pressurization system configured to generate a pressure in the at least one first cavity 29 and the at least one second cavity 30. In some embodiments, the pump may be connected to a dialysis machine, such as, but not limited to, a hemodialysis machine, a peritoneal dialysis machine, a hemofiltration machine, a hemodiafiltration machine, or an intestinal dialysis machine. In other embodiments, the pump may be connected to a heart-lung machine.

In an embodiment, the fluid pump system 64 is used as a bubble trap, i.e. the direction of flow from the first cavity 29 to the second cavity 30 is downwards and any bubbles are thus trapped in the first cavity 29 One option of emptying the bubbles is shown in FIG. 7 by the draining tube 10.

Another embodiment of the present invention may provide a method of pumping a biological fluid 7 to a target 5, the method including providing a pump system 64. The pump system 64 may comprise a fluid reservoir 21 comprising a cavity 29 at least partially defined by a deformable membrane 33 and/or bag 28 to provide a volume; a fluid-container system 27 in fluid communication with the fluid reservoir 21; and a pressurization system 1, 2, 58, 59, 60, 61 configured to generate a pressure in the cavity 29. A first pressure may be generated by the method and may be configured to deform the membrane 33 and/or bag 28 to expand the volume in the cavity 29. The membrane 33 and/or bag 28 may be deformed using the pressurization system 1, 2, 58, 59, 60, 61. By expanding the volume available in the cavity 29, the biological fluid 7 may flow from the fluid-container system 27 into the cavity 29. A second pressure may be generated by the method and may be configured to deform the membrane 33 and/or bag 28 to reduce the volume of said cavity. By reducing the volume of the cavity 29, the biological fluid 7 may flow to the target 5.

As aspect of the embodiments therefore relates to a method of pumping a biological fluid 7 to a target 5. The method comprises providing a fluid pump system 64 comprising a fluid reservoir 21 comprising at least one first cavity 29 and at least one second cavity 30 in fluid communication with each other and configured to accommodate the biological fluid 7, wherein the at least one second cavity 30 is in fluid communication with the target 5. The fluid pump system 64 also comprises a fluid-container system 27 in fluid communication with the at least one first cavity 29 in the fluid reservoir 21 and a pressurization system 1, 2, 58, 59, 60, 61 configured to generate a pressure in the at least one first cavity 29 and the at least one second cavity 30. The method also comprises generating a first pressure below ambient pressure in the at least one first cavity 29 using the pressurization system 1, 2, 58, 59, 60, 61, thereby causing the biological fluid 7 to flow from the fluid-container system 27 into the at least one first cavity 29. The method further comprises generating a second pressure in the at least one first cavity 29 using the pressurization system 1, 2, 58, 59, 60, 61, thereby causing the biological fluid 7 in the at least one first cavity 29 to flow into the at least one second cavity 30. The method additionally comprises applying the second pressure to the at least one second cavity 30, thereby pumping the biological fluid 7 to the target 5.

In an embodiment, applying the second pressure comprises constantly applying the second pressure to the at least one second cavity 30.

In an embodiment, applying the second pressure comprises applying the second pressure to the at least one second cavity 30 to provide the same external pressure on the at least one first cavity 29 and the at least one second cavity 30.

In an embodiment, applying the second pressure comprises applying the second pressure to the at least one second cavity 30 before the second pressure is applied to the at least one first cavity 29.

In an embodiment, applying the second pressure comprises applying the second pressure to both the at least one first cavity 29 and the at least one second cavity 30 during a time period.

In an embodiment, the method further comprises flowing the biological fluid 7 in the at least one first cavity 29 by gravity into the at least one second cavity 30.

In an embodiment, the method further comprises applying the first pressure as a substantially constant or pulsatile pressure below ambient pressure to the at least one first cavity 29. In this embodiment, applying the second pressure comprises applying the second pressure as a substantially constant or pulsatile pressure to the at least one second cavity 30.

In an embodiment, generating the first pressure comprises generating the first pressure as substantially constant or pulsatile pressure below ambient pressure using the pressurization system 1, 2, 58, 59, 60, 61. In this embodiment, generating the second pressure comprises generating the second pressure as a substantially constant or pulsatile pressure using the pressurization system 1, 2, 58, 59, 60, 61.

In an embodiment, the at least one first cavity 29 comprises a first membrane 33, thereby forming a first chamber 31 and a second chamber 32 and/or the at least one second cavity 30 comprises a second membrane 43, thereby forming a third chamber 41 and a fourth chamber 42.

In an embodiment, the at least one first cavity 29 comprises a first bag 65, thereby forming a first chamber 31 and a second chamber 32 and/or the at least one second cavity 30 comprises a second bag 66, thereby forming a third chamber 41 and a fourth chamber 42.

In an embodiment, the first membrane 33 and/or the second membrane 43 or the first bag 65 and/or the second bag 66 comprise a polymeric material.

In an embodiment, the method further comprises adding oxygen through the first membrane 33 or the first bag 65 and/or the second membrane 43 or the second bag 66.

In an embodiment, the method further comprises degassing the biological fluid 7, optionally wherein the biological fluid 7 is degassed using a first membrane 33 of the at least one first cavity 29, or a first bag 65 of the at least one first cavity 29.

In an embodiment, the method further comprises detecting a first deformed position 54 of the first membrane 33 or the first bag 65 prior to generating the second pressure in the at least one first cavity 29.

In an embodiment, detecting the first deformed position 54 comprises detecting the first deformed position 54 of the first membrane 33 or the first bag 65 using a sensor.

In an embodiment, the method, i.e. the fluid pump system 64, is configured to provide a shear force of less than about 15 Pa on the biological fluid 7.

In an embodiment, the method, i.e. the fluid pump system 64, is configured to provide a pressure stability of less than about 5% on the biological fluid 7.

In an embodiment, applying the second pressure comprises applying the second pressure to the at least one second cavity 30, thereby pumping an amount of the biological fluid 7 to the target 5 of about 2 millilitre/min to about 10 litre/min.

In an embodiment, applying the second pressure comprises applying the second pressure to the at least one second cavity 30, thereby pressurizing the biological fluid 7 pumped to the target 5 to about 2 mmHg to about 130 mmHg.

In an embodiment, the fluid pump system 64 comprises a pump for pumping a biological fluid comprising a protein, an antibody, a biological or complex drug, a vaccine, a therapeutic agent and/or a biopharmaceutical, a starting material and/or intermediate for production of the protein, the antibody, the biological or complex drug, the vaccine, the therapeutic agent and/or the biopharmaceutical, a cell and any combination thereof.

In an embodiment, wherein the fluid pump system 64 comprises an extracorporeal blood pump.

In an embodiment, the target 5 is a dialysis machine.

In an embodiment, the biological fluid is selected from a group consisting of blood, serum, a fluid comprising a blood component, a fluid comprising a cell, a cell culture fluid, a fluid comprising at least one of a protein, an antibody, a biological or complex drug, a vaccine, a therapeutic agent, a biopharmaceutical, and/or a starting material and/or intermediate for production of the protein, the antibody, the biological or complex drug, the vaccine, the therapeutic agent and/or the biopharmaceutical.

Another aspect of the embodiments relates to a fluid pump system 64 comprising a fluid reservoir 21 comprising at least one first cavity 29 and at least one second cavity 30 in fluid communication with each other and configured to accommodate a biological fluid 7. The fluid pump system 64 also comprises a fluid-container system 27 in fluid communication with the at least one first cavity 29 in the fluid reservoir 21. The fluid pump system 64 further comprises a pressurization system 1, 2, 58, 59, 60, 61 configured to generate a first pressure below ambient pressure in the at least one first cavity 29, thereby causing the biological fluid 7 to flow from the fluid-container system 27 to the at least one first cavity 29. The pressurization system 1, 2, 58, 59, 60, 61 is also configured to generate a second pressure in the at least one first cavity 29, thereby causing the biological fluid 7 in the at least one first cavity 29 to flow into the at least one second cavity 30. The pressurization system 1, 2, 58, 59, 60, 61 is further configured to apply the second pressure to the at least one second cavity 30, thereby pumping the biological fluid 7 out from the at least one second cavity 30.

In an embodiment, the fluid pump system 64 is an extracorporeal blood pump system, the fluid reservoir 21 comprises the at least one first cavity 29 and the at least one second cavity 30 in fluid communication with each other and configured to accommodate blood 7, and the pressurization system 1, 2, 58, 59, 60, 61 is configured to generate the first pressure below ambient pressure in the at least one first cavity 29, thereby causing the blood 7 to flow from the fluid-container system 27 to the at least one first cavity 27, generate the second pressure in the at least one first cavity 29, thereby causing the blood 7 in the at least one first cavity 29 to flow into the at least one second cavity 30, and apply the second pressure to the at least one second cavity 30, thereby pumping the blood 7 out from the at least one second cavity 30.

In an embodiment, the fluid pump system 64 further comprises a dialysis machine connected to the at least one first cavity 29 and/or the at least one second cavity 30.

In an embodiment, the dialysis machine is selected from a group consisting of a hemodialysis machine, a peritoneal dialysis machine, a hemofiltration machine, a hemodiafiltration machine, an intestinal dialysis machine.

In an embodiment, the fluid pump system 64 further comprises a heart-lung machine connected to the at least one first cavity 29 and/or the at least one second cavity 30.

In an embodiment, fluid pump system 64 further comprises a target 5 connected to the at least one second cavity 30, the biological fluid is selected from a group consisting of blood, serum, a fluid comprising a blood component, a fluid comprising a cell, a fluid comprising at least one of a protein, an antibody, a biological or complex drug, a vaccine, a therapeutic agent, a biopharmaceutical, and/or a starting material and/or intermediate for production of the protein, the antibody, the biological or complex drug, the vaccine, the therapeutic agent and/or the biopharmaceutical.

In an embodiment, the pressurization system 1, 2, 58, 59, 60, 61 is configured to constantly apply the second pressure to the at least one second cavity 30.

In an embodiment, the at least one first cavity 29 comprises a first membrane 33, thereby forming a first chamber 31 and a second chamber 32 and/or the at least one second cavity 30 comprises a second membrane 43, thereby forming a third chamber 41 and a fourth chamber 42.

In an embodiment, the at least one first cavity 29 comprises a first bag 65, thereby forming a first chamber 31 and a second chamber 32 and/or the at least one second cavity 30 comprises a second bag 66, thereby forming a third chamber 41 and a fourth chamber 42.

In an embodiment, the first membrane 33 and/or the second membrane 43 or the first bag 65 and/or the second bag 66 comprise a polymeric material.

In an embodiment, the first membrane 33 and/or first bag 65 is permeable to at least one gas and/or the second membrane 43 and/or second bag 66 is permeable to at least one gas.

In an embodiment, the at least one gas is oxygen.

In an embodiment, the fluid pump system 64 is configured to provide a shear force of less than about 15 Pa on the biological fluid.

In an embodiment, the first pressure and the second pressure are substantially constant.

In an embodiment, biological fluid in the at least one first cavity 29 flows by gravity into the at least one second cavity 30.

In an embodiment, the pressurization system 1, 2, 58, 59, 60, 61 is configured to provide a substantially constant pressure using air.

In an embodiment, the pressurization system 1, 2, 58, 59, 60, 61 is further configured to provide a vacuum using air.

In an embodiment, the fluid pump system 64 further comprises a vacuum pump configured to provide the vacuum using air.

The specifications of dimensions and materials given in the descriptions are not intended as characteristics and should not be construed as limitations of the invention.

### EXAMPLES

### Test of Pumping Blood

The aim was to investigate the performance of the pump with regard to the mashing of red blood cells. We let the very same blood pass several times through a fluid pump system i.e. up to approximately 34 times. The setup was according to the embodiments shown in FIG. 9 where the fluid-container system 27 was in the form of an open tray and the target 5 the very same tray. The blood used in the experiment was derived from adult pigs.

We took about 400 ml of fresh pig blood and put in an open tray and covered the tray with a plastic film in order to reduce clotting. The first hose 12, i.e. the blood inlet, was inserted in the tray and the second tube 4, i.e. the blood outlet, was returned to the tray and the flow was set to approximately 75 ml/min. Before and at specific times of pumping we observed the blood in a microscope to track the status of the red blood cells, see Table 1.

TABLE 1
Status of red blood cells

| Time | No of passes | Degradation of red blood cells |
|---|---|---|
| 0 | 0 | Good |

| | | | |
|---|---|---|---|
| 5 | s | 1 | No visual difference (NVD) |
| 10 | min | 2 | NVD |
| 20 | min | 4 | NVD |
| 30 | min | 6 | NVD |
| 60 | min | 11 | NVD |
| 120 | min | 23 | NVD |
| 180 | min | 34 | NVD |

We let the test run further than 180 min but clotting began to arise on the surface and the test was discontinued.

The conclusion is that we did not observe any degradation of the red bloods cells within the range of 34 passes through the fluid pump system, supporting the fact that the fluid pump system is suitable for pumping blood and other delicate biological fluids.

Bioreactor Test

The aim was to investigate the performance of a fluid pump system of the embodiments with regards to providing a specific pressure and flow and formation of bubbles to a bioreactor made for tissue engineering. The flow and pressure was set to be adapted for the organ liver, approximately a desired pressure of 5-10 mmHg and flow of 55 mL/min.

In the bioreactor, one first remove all the cells from an organ or tissue by decellularization where the donor cells are stripped away and a protein scaffold is left behind. The next step can be to repopulate the scaffold again—recellularization, by using e.g. autologous stem cells from the recipient, i.e. immunologically matched cells.

Our analysis was to simulate the step of decellularization of a liver using a 2% SDC (sodium deoxycholate) solution and saline as fluids with the above mentioned pressures and flow. In order to simulate the liver and its flow resistance a two-way valve was used. The pressure on the pump was set to approximately 9 mmHg and the valve was regulated to give a flow of approximately 55 mL/min.

The conclusion was that we did not observe any bubbles out from the pump and we were able to supply the desired pressure and flow and thereby supporting the fact that the fluid pump system is suitable for pumping detergents, saline and other delicate biological fluids, e.g. for use together with a bioreactor used for production of tissue/organs.

1. A method of pumping a biological fluid to a target said method comprising:
providing a fluid pump system comprising:
a fluid reservoir comprising at least one first cavity and at least one second cavity in fluid communication with each other and configured to accommodate said biological fluid, wherein said at least one second cavity is in fluid communication with said target;
a fluid-container system in fluid communication with said at least one first cavity in said fluid reservoir; and
a pressurization system configured to generate a pressure in said at least one first cavity and said at least one second cavity;
generating a first pressure below ambient pressure in said at least one first cavity using said pressurization system, thereby causing said biological fluid to flow from said fluid-container system into said at least one first cavity;
generating a second pressure in said at least one first cavity using said pressurization system, thereby causing said biological fluid in said at least one first cavity to flow into said at least one second cavity; and
applying said second pressure to said at least one second cavity, thereby pumping said biological fluid to said target.

2. The method of claim 1, wherein applying said second pressure comprises constantly applying said second pressure to said at least one second cavity.

3. The method of claim 1, wherein applying said second pressure comprises applying said second pressure to said at least one second cavity to provide the same external pressure on said at least one first cavity and said at least one second cavity.

4.-5. (canceled)

6. The method of claim 1, further comprising flowing said biological fluid in said at least one first cavity by gravity into said at least one second cavity.

7. The method of claim 1, further comprising:
applying said first pressure as a substantially constant or pulsatile pressure below ambient pressure to said at least one first cavity, wherein
applying said second pressure comprises applying said second pressure as a substantially constant or pulsatile pressure to said at least one second cavity.

8. (canceled)

9. The method of claim 1, wherein said at least one first cavity comprises a first membrane, thereby forming a first chamber and a second chamber and/or said at least one second cavity comprises a second membrane, thereby forming a third chamber and a fourth chamber.

10. The method of claim 1, wherein said at least one first cavity comprises a first bag, thereby forming a first chamber and a second chamber and/or said at least one second cavity comprises a second bag, thereby forming a third chamber and a fourth chamber.

11.-16. (canceled)

17. The method of claim 1, wherein said method is configured to provide a pressure stability of less than about 5% on said biological fluid.

18. The method of claim 1, wherein applying said second pressure comprises applying said second pressure to said at least one second cavity to pump said biological fluid to said target in an amount of about 2 mL/min to about 10 L/min.

19. The method of claim 1, wherein applying said second pressure comprises applying said second pressure to said at least one second cavity to pressurize said biological fluid pumped to said target to about 2 mmHg to about 130 mmHg.

20. The method of claim 1, wherein said fluid pump system comprises a pump for pumping a biological fluid comprising a protein, an antibody, a biological or complex drug, a vaccine, a therapeutic agent and/or a biopharmaceutical, a starting material and/or intermediate for production of said protein, said antibody, said biological or complex drug, said vaccine, said therapeutic agent and/or said biopharmaceutical, a cell, a cell culture medium and any combination thereof.

21. The method of claim 1, wherein said fluid pump system comprises an extracorporeal blood pump.

22. The method of claim 1, wherein said target is a dialysis machine.

23. The method of claim 1, wherein said biological fluid is selected from a group consisting of blood, serum, a fluid comprising a blood component, a cell culture medium, a fluid comprising a cell, a fluid comprising at least one of a protein, an antibody, a biological or complex drug, a vaccine, a therapeutic agent, a biopharmaceutical, and/or a starting material and/or intermediate for production of said protein, said antibody, said biological or complex drug, said vaccine, said therapeutic agent and/or said biopharmaceutical.

24. A fluid pump system comprising:
a fluid reservoir comprising at least one first cavity and at least one second cavity in fluid communication with each other and configured to accommodate a biological fluid;
a fluid-container system in fluid communication with said at least one first cavity in said fluid reservoir; and
a pressurization system configured to:
generate a first pressure below ambient pressure in said at least one first cavity, thereby causing said biological fluid to flow from said fluid-container system to said at least one first cavity,
generate a second pressure in said at least one first cavity, thereby causing said biological fluid in said at least one first cavity to flow into said at least one second cavity, and
apply said second pressure to said at least one second cavity, thereby pumping said biological fluid out from said at least one second cavity.

25. The fluid pump system of claim 24, wherein
said fluid pump system is an extracorporeal blood pump system;
said fluid reservoir comprises said at least one first cavity and said at least one second cavity in fluid communication with each other and configured to accommodate blood; and
said pressurization system is configured to:
generate said first pressure below ambient pressure in said at least one first cavity, thereby causing said blood to flow from said fluid-container system to said at least one first cavity,
generate said second pressure in said at least one first cavity, thereby causing said blood in said at least one first cavity to flow into said at least one second cavity, and
apply said second pressure to said at least one second cavity, thereby pumping said blood out from said at least one second cavity.

26. The fluid pump system of claim 24, further comprising a dialysis machine connected to said at least one first cavity and/or said at least one second cavity.

27. (canceled)

28. The fluid pump system of claim 24, further comprising a heart-lung machine connected to said at least one first cavity and/or said at least one second cavity.

29. The fluid pump system according to claim 24, further comprising a target connected to said at least one second cavity, said biological fluid is selected from a group consisting of blood, serum, a cell culture medium, a fluid comprising a blood component, a fluid comprising a cell, a fluid comprising at least one of a protein, an antibody, a biological or complex drug, a vaccine, a therapeutic agent, a biopharmaceutical, and/or a starting material and/or intermediate for production of said protein, said antibody, said biological or complex drug, said vaccine, said therapeutic agent and/or said biopharmaceutical,
wherein the target is a machine, laboratory consumable, device for use with the biological fluid, in vitro organ and/or tissue, and/or ex vivo organ and/or tissue.

30. The fluid pump system of claim 24, wherein said pressurization system is configured to constantly apply said second pressure to said at least one second cavity.

31. The fluid pump system of claim 24, wherein said at least one first cavity comprises a first membrane, thereby forming a first chamber and a second chamber and/or said at least one second cavity comprises a second membrane, thereby forming a third chamber and a fourth chamber.

32. The fluid pump system of claim 24, wherein said at least one first cavity comprises a first bag, thereby forming a first chamber and a second chamber and/or said at least one second cavity comprises a second bag, thereby forming a third chamber and a fourth chamber.

33. (canceled)

34. The fluid pump system of claim 31, wherein said first membrane and/or first bag is permeable to at least one gas and/or said second membrane and/or second bag is permeable to at least one gas.

35. (canceled)

36. The fluid pump system of claim 24, wherein said fluid pump system is configured to provide a shear force of less than about 15 Pa on said biological fluid.

37. (canceled)

38. The fluid pump system of claim 24, wherein said biological fluid in said at least one first cavity flows by gravity into said at least one second cavity.

39.-41. (canceled)

42. The method of claim 10, wherein the first bag and/or second bag is a tube-like bag.

43. The method of claim 1, wherein said at least one first cavity comprises a first bag and first membrane and said at least one second cavity comprises a second bag and second membrane, and wherein said first membrane and/or first bag is permeable to at least one gas and/or said second membrane and/or second bag is permeable to at least one gas.

44. The method of claim 1, further comprising:
applying said first pressure as a substantially constant or pulsatile pressure below ambient pressure to said at least one first cavity, and wherein
applying said second pressure to said at least one second cavity comprises applying said second pressure as a pulsatile pressure.

45. The fluid pump system of claim 24, wherein said pressurization system is configured to apply said second pressure to said at least one second cavity, and said second pressure is a pulsatile pressure.

46. The fluid pump system of claim 24, further comprising a draining tube in fluid communication with said at least one first cavity.

47. The fluid pump system of claim 24, wherein one or more parts of the fluid pump system is single use.

48. The fluid pump system of claim 32, wherein the first bag and/or second bag is a tube-like bag.

49. The fluid pump system of claim 24, wherein said pressurization system is configured to apply said second pressure to said at least one second cavity and pump said biological fluid to a target in an amount of about 2 mL/min to about 10 L/min.

50. The fluid pump system of claim 24, wherein said pressurization system is configured to apply said second pressure to said at least one second cavity and pressurize said biological fluid to about 2 mmHg to about 130 mmHg.
